(19) United States
(12) Patent Application Publication
(10) Pub. No.: US 2008/0096929 A1
(43) Pub. Date: Apr. 24, 2008

(54) Novel Use

(75) Inventors: Anna-Karin Tiden, Sodertalje (SE); Dominika Turek, Sodertalje (SE); Jenny Viklund, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 11/577,833

(22) PCT Filed: Oct. 24, 2005
(86) PCT No.: PCT/SE05/01593

§ 371 Date: Apr. 24, 2007

(30) Foreign Application Priority Data
Oct. 25, 2004 (SE) 0402591-2

(51) Int. Cl. A61K 31/4196; A61K 31/4439; A61P 29/00; C07D 249/10; C07D 401/06

(52) U.S. Cl. 514/340; 514/384; 546/272.4; 548/263.2

(57) ABSTRACT

There is disclosed the use of a compound of formula (I), (I) wherein X, Y, W and Q are as defined in the specification, and pharmaceutically acceptable salts thereof, in the manufacture of a medicament, for the treatment or prophylaxis of diseases or conditions in which inhibition of the enzyme myeloperoxidase (MPO) is beneficial. Certain novel compounds of formula (I) and pharmaceutically acceptable salts thereof are disclosed, together with processes for their preparation. The compounds of formulae (I) are MPO inhibitors and are thereby particularly useful in the treatment or prophylaxis of neuroinflammatory disorders.

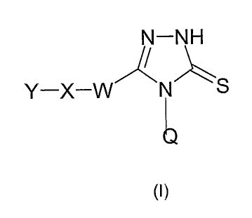

### FIELD OF THE INVENTION

The present invention relates to the use of derivatives of 2,4-dihydro-[1,2,4]triazole-3-thione as inhibitors of the enzyme myeloperoxidase (MPO). Certain novel 2,4-dihydro-[1,2,4]triazole-3-thione derivatives are also disclosed together with processes for their preparation, compositions containing them and their use in therapy.

### BACKGROUND OF THE INVENTION

Myeloperoxidase (MPO) is a heme-containing enzyme found predominantly in polymorphonuclear leukocytes (PMNs). MPO is one member of a diverse protein family of mammalian peroxidases that also includes eosinophil peroxidase, thyroid peroxidase, salivary peroxidase, lactoperoxidase, prostaglandin H synthase, and others. The mature enzyme is a dimer of identical halves. Each half molecule contains a covalently bound heme that exhibits unusual spectral properties responsible for the characteristic green colour of MPO. Cleavage of the disulphide bridge linking the two halves of MPO yields the hemi-enzyme that exhibits spectral and catalytic properties indistinguishable from those of the intact enzyme. The enzyme uses hydrogen peroxide to oxidize chloride to hypochlorous acid. Other halides and pseudohalides (like thiocyanate) are also physiological substrates to MPO.

PMNs are of particular importance for combating infections. These cells contain MPO, with well documented microbicidal action. PMNs act non-specifically by phagocytosis to engulf microorganisms, incorporate them into vacuoles, termed phagosomes, which fuse with granules containing myeloperoxidase to form phagolysosomes. In phagolysosomes the enzymatic activity of the myeloperoxidase leads to the formation of hypochlorous acid, a potent bactericidal compound. Hypochlorous acid is oxidizing in itself, and reacts most avidly with thiols and thioethers, but also converts amines into chloramines, and chlorinates aromatic amino acids. Macrophages are large phagocytic cells which, like PMNs, are capable of phagocytosing microorganisms. Macrophages can generate hydrogen peroxide and upon activation also produce myeloperoxidase. MPO and hydrogen peroxide can also be released to the outside of the cells where the reaction with chloride can induce damage to adjacent tissue.

Linkage of myeloperoxidase activity to disease has been implicated in neurological diseases with a neuroinflammatory response including multiple sclerosis, Alzheimer's disease, Parkinson's disease and stroke as well as other inflammatory diseases or conditions like asthma, chronic obstructive pulmonary disease, cystic fibrosis, atherosclerosis, inflammatory bowel disease, renal glomerular damage and rheumatoid arthritis. Lung cancer has also been suggested to be associated with high MPO levels.

WO 01/85146 discloses various compounds that are MPO inhibitors and are thereby useful in the treatment of chronic obstructive pulmonary disease (COPD).

The present invention relates to a group of 2,4-dihydro-[1,2,4]triazole-3-thione derivatives that surprisingly display useful properties as inhibitors of the enzyme MPO.

### DISCLOSURE OF THE INVENTION

According to the present invention, there is provided the use of a compound of formula (I) 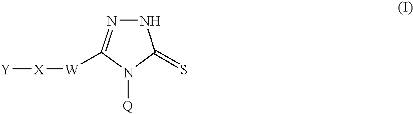
wherein:
Q represents a 5 to 7-membered saturated or partially unsaturated heterocyclic ring containing one or two heteroatoms independently selected from O, NR¹⁴ and S; said heterocyclic ring being optionally substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³ or NR⁴R⁵; said alkyl, cycloalkyl, alkoxy and alkylthio groups being optionally further substituted by phenyl or by one or more halogen atoms;
or Q represents C3 to 8 cycloalkyl substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³ or NR⁴R⁵; said alkyl, C3 to 6 cycloalkyl, alkoxy and alkylthio groups being optionally further substituted by phenyl or by one or more halogen atoms;
or Q represents partially unsaturated C5 to 8 cycloalkyl optionally substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³ or NR⁴R⁵; said alkyl, C3 to 6 cycloalkyl, alkoxy and alkylthio groups being optionally further substituted by phenyl or by one or more halogen atoms;
or Q represents saturated or partially unsaturated C6 to 8 bicycloalkyl optionally substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³ or NR⁴R⁵; said alkyl, C3 to 6 cycloalkyl, alkoxy and alkylthio groups being optionally further substituted by phenyl or by one or more halogen atoms;
and in each of the above definitions the ring Q is optionally benzo fused wherein the benzo ring is optionally substituted by one or more substituents independently selected from halogen, CHO, C2 to 6 alkanoyl, C1 to 6 alkyl, C1 to 6 alkylthio and C1 to 6 alkoxy;
or Q represents benzo fused C4 to 8 cycloalkyl wherein the benzo ring is optionally substituted by one or more substituents independently selected from halogen, CHO, C2 to 6 alkanoyl, C1 to 6 alkyl, C1 to 6 alkylthio and C1 to 6 alkoxy;
W represents a bond or CHR¹ wherein R¹ represents H, CH₃, F, OH, CH₂OH or phenyl;
X represents a bond, O, CH₂ or NR³ wherein R³ represents H or C1 to 6 alkyl;
Y represents phenyl, naphthyl or a monocyclic or bicyclic heteroaromatic ring system containing one to three heteroatoms independently selected from O, N and S; said phenyl, naphthyl or heteroaromatic ring system being optionally substituted by one to three substituents independently selected from halogen, OH, C1 to 6 alkyl, C3 to 6 cycloalkyl, C1 to 6 alkoxy, C1 to 6 alkylthio, CO₂H, C2 to 6 alkanoyl, phenyl, NO₂, C(O)NR¹²R¹³ or NR⁴R⁵; said alkyl, cycloalkyl, alkoxy and alkylthio groups being optionally further substituted by one or more fluoro atoms;
or Y represents C1 to 6 alkyl or C3 to 6 cycloalkyl; said cycloalkyl group optionally including an O atom and optionally being benzo fused; and said alkyl or cycloalkyl group being optionally substituted by one or more substituents independently selected from halogen, oxo (═O), C1 to 6 alkyl or C1 to 6 alkoxy;
each R⁴, R⁵, R⁶, R¹² and R¹³ independently represents H or C1 to 6 alkyl;
R¹⁴ represents H, C1 to 6 alkyl, CHO or C2 to 6 alkanoyl; said alkyl being optionally further substituted by phenyl wherein the phenyl group may be optionally further substituted by halogen, C1 to 6 alkyl, C1 to 6 alkoxy or C2 to 6 alkanoyl;
or a pharmaceutically acceptable salt thereof, in the manufacture of a medicament, for the treatment or prophylaxis of diseases or conditions in which inhibition of the enzyme MPO is beneficial.

The compounds of formula (I) may exist in enantiomeric forms. Therefore, all enantiomers, diastereomers, racemates and mixtures thereof are included within the scope of the invention.

The compounds of formula (I) may exist in tautomeric forms. All such tautomers and mixtures of tautomers are included within the scope of the present invention.

A more particular aspect of the invention provides the use of a compound of formula (I), or a pharmaceutically acceptable salt thereof, in the manufacture of a medicament, for the treatment or prophylaxis of neuroinflammatory disorders.

According to the invention, there is also provided a method of treating, or reducing the risk of, diseases or conditions in which inhibition of the enzyme MPO is beneficial which comprises administering to a person suffering from or at risk of, said disease or condition, a therapeutically effective amount of a compound of formula (I), or a pharmaceutically acceptable salt thereof.

More particularly, there is also provided a method of treating, or reducing the risk of, neuroinflammatory disorders in a person suffering from or at risk of, said disease or condition, wherein the method comprises administering to the person a therapeutically effective amount of a compound of formula (I), or a pharmaceutically acceptable salt thereof.

In another aspect the invention provides a pharmaceutical formulation comprising a therapeutically effective amount of a compound of formula (I), or a pharmaceutically acceptable salt thereof, in admixture with a pharmaceutically acceptable adjuvant, diluent or carrier, for use in the treatment or prophylaxis of diseases or conditions in which inhibition of the enzyme MPO is beneficial.

In another more particular aspect the invention provides a pharmaceutical formulation comprising a therapeutically effective amount of a compound of formula (I), or a pharmaceutically acceptable salt thereof, in admixture with a pharmaceutically acceptable adjuvant, diluent or carrier, for use in the treatment or prophylaxis of neuroinflammatory disorders.

In one embodiment, Q represents an optionally benzo fused 5 to 7-membered saturated or partially unsaturated heterocyclic ring containing one or two heteroatoms independently selected from O, NR¹⁴ and S; said heterocyclic ring being optionally substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³ or NR⁴R⁵; said alkyl, cycloalkyl, alkoxy and alkylthio groups being optionally further substituted by phenyl or by one or more halogen atoms; and X, Y and W are as defined above.

In one embodiment, Q represents an optionally benzo fused C3 to 8 cycloalkyl substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³ or NR⁴R⁵; said alkyl, C3 to 6 cycloalkyl, alkoxy and alkylthio groups being optionally further substituted by phenyl or by one or more halogen atoms; and X, Y and W are as defined above.

In one embodiment, Q represents an optionally benzo fused partially unsaturated C5 to 8 cycloalkyl optionally substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³ or NR⁴R⁵; said alkyl, C3 to 6 cycloalkyl, alkoxy and alkylthio groups being optionally further substituted by phenyl or by one or more halogen atoms; and X, Y and W are as defined above.

In one embodiment, Q represents an optionally benzo fused saturated or partially unsaturated C6 to 8 bicycloalkyl optionally substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³ or NR⁴R⁵; said alkyl, C3 to 6 cycloalkyl, alkoxy and alkylthio groups being optionally further substituted by phenyl or by one or more halogen atoms; and X, Y and W are as defined above.

In one embodiment, Q represents benzo fused C4 to 8 cycloalkyl wherein the benzo ring is optionally substituted by one or more substituents independently selected from halogen, CHO, C2 to 6 alkanoyl, C1 to 6 alkyl, C1 to 6 alkylthio and C1 to 6 alkoxy; and X, Y and W are as defined above.

In one embodiment, W represents a bond or CH₂.

In one embodiment, X represents a bond or O.

In one embodiment, W represents CH₂ and X represents a bond.

In one embodiment, W represents CH₂ and X represents O.

In one embodiment, Y represents phenyl optionally substituted as defined above.

In one embodiment, Y represents pyridyl optionally substituted as defined above. In one embodiment, Y represents 2-pyridyl optionally substituted as defined above.

In one embodiment, Q represents an optionally benzo fused 5 to 7-membered saturated or partially unsaturated heterocyclic ring containing one or two heteroatoms independently selected from O, NR¹⁴ and S; said heterocyclic ring being optionally substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³ or NR⁴R⁵; said alkyl, cycloalkyl, alkoxy and alkylthio groups being optionally further substituted by phenyl or by one or more halogen atoms; W represents CH₂; X represents O; and Y represents phenyl or pyridyl optionally substituted as defined above.

In one embodiment, Q represents an optionally benzo fused 5 to 7-membered saturated or partially unsaturated heterocyclic ring containing one or two heteroatoms independently selected from O, NR¹⁴ and S; said heterocyclic ring being optionally substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³ or NR⁴R⁵; said alkyl, cycloalkyl, alkoxy and alkylthio groups being optionally further substituted by phenyl or by one or more halogen atoms; W represents CH₂; X represents a bond; and Y represents phenyl or pyridyl optionally substituted as defined above.

In one embodiment, Q represents an optionally benzo fused C3 to 8 cycloalkyl substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³ or NR⁴R⁵; said alkyl, C3 to 6 cycloalkyl, alkoxy and alkylthio groups being optionally further substituted by phenyl or by one or more halogen atoms; W represents CH₂; X represents O; and Y represents phenyl or pyridyl optionally substituted as defined above.

In one embodiment, Q represents an optionally benzo fused C3 to 8 cycloalkyl substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³ or NR⁴R⁵; said alkyl, C3 to 6 cycloalkyl, alkoxy and alkylthio groups being optionally further substituted by phenyl or by one or more halogen atoms; W represents CH₂; X represents a bond; and Y represents phenyl or pyridyl optionally substituted as defined above.

In one embodiment, Q represents an optionally benzo fused partially unsaturated C5 to 8 cycloalkyl optionally substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³ or NR⁴R⁵; said alkyl, C3 to 6 cycloalkyl, alkoxy and alkylthio groups being optionally further substituted by phenyl or by one or more halogen atoms; W represents CH₂; X represents O; and Y represents phenyl or pyridyl optionally substituted as defined above.

In one embodiment, Q represents an optionally benzo fused partially unsaturated C5 to 8 cycloalkyl optionally substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³ or NR⁴R⁵; said alkyl, C3 to 6 cycloalkyl, alkoxy and alkylthio groups being optionally further substituted by phenyl or by one or more halogen atoms; W represents CH₂; X represents a bond; and Y represents phenyl or pyridyl optionally substituted as defined above.

In one embodiment, Q represents an optionally benzo fused saturated or partially unsaturated C6 to 8 bicycloalkyl optionally substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³ or NR⁴R⁵; said alkyl, C3 to 6 cycloalkyl, alkoxy and alkylthio groups being optionally further substituted by phenyl or by one or more halogen atoms; W represents CH₂; X represents O; and Y represents phenyl or pyridyl optionally substituted as defined above.

In one embodiment, Q represents an optionally benzo fused saturated or partially unsaturated C6 to 8 bicycloalkyl optionally substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³ or NR⁴R⁵; said alkyl, C3 to 6 cycloalkyl, alkoxy and alkylthio groups being optionally further substituted by phenyl or by one or more halogen atoms; W represents CH₂; X represents a bond; and Y represents phenyl or pyridyl optionally substituted as defined above.

In one embodiment, Q represents benzo fused C4 to 8 cycloalkyl wherein the benzo ring is optionally substituted by one or more substituents independently selected from halogen, CHO, C2 to 6 alkanoyl, C1 to 6 alkyl, C1 to 6 alkylthio and C1 to 6 alkoxy; W represents CH₂; X represents O; and Y represents phenyl or pyridyl optionally substituted as defined above.

In one embodiment, Q represents benzo fused C4 to 8 cycloalkyl wherein the benzo ring is optionally substituted by one or more substituents independently selected from halogen, CHO, C2 to 6 alkanoyl, C1 to 6 alkyl, C1 to 6 alkylthio and C1 to 6 alkoxy; W represents CH₂; X represents a bond; and Y represents phenyl or pyridyl optionally substituted as defined above.

A specific aspect of the invention concerns the use of any one or more of the following compounds of formula (I):
5-phenoxymethyl-4-(dihydrofuran-2-one-3-yl)-2,4-dihydro-[1,2,4]triazole-3-thione;
5-(2-chloro-phenoxymethyl)-4-(trans-2-hydroxycyclohexyl)-2,4-dihydro-[1,2,4]triazole-3-thione;
5-phenoxymethyl-4-(1,2,3,4-tetrahydro-naphthalen-1-yl)-2,4-dihydro-[1,2,4]triazole-3-thione;
4-(bicyclo[2.2.1]hept-5-en-2-yl)-5-pyridin-2-ylmethyl-2,4-dihydro-[1,2,4]triazole-3-thione;
4-(1-benzyl-pyrrolidin-3-yl)-5-pyridin-2-ylmethyl-2,4-dihydro-[1,2,4]triazole-3-thione;
4-((1R,2R)-2-benzyloxy-cyclopentyl)-5-pyridin-2-ylmethyl-2,4-dihydro-[1,2,4]triazole-3-thione;
   or a pharmaceutically acceptable salt thereof.

Unless otherwise indicated, the term “C1 to 6 alkyl” referred to herein denotes a straight or branched chain alkyl group having from 1 to 6 carbon atoms. Examples of such groups include methyl, ethyl, 1-propyl, n-butyl, iso-butyl, tert-butyl, pentyl and hexyl. The term “C1 to 2 alkyl” is to be interpreted analogously.

Unless otherwise indicated, the term “C3 to 8 cycloalkyl” referred to herein denotes a cyclic alkyl group having from 3 to 8 carbon atoms. Examples of such groups include cyclopropyl, cyclopentyl and cyclohexyl. The term “C3 to 6 cycloalkyl” is to be interpreted analogously. The term “C3 to 6 cycloalkyl; said cycloalkyl group optionally including an O atom and optionally being benzo fused” is to be interpreted analogously. Examples of such groups include tetrahydrofuran, oxane, indan, tetrahydronaphthalene, chroman and isochroman,

Unless otherwise indicated, the term “unsaturated C5 to 8 cycloalkyl” referred to herein denotes a cyclic alkyl group having from 5 to 8 carbon atoms and incorporating one or more double bonds. Examples of such groups include cyclopentenyl, cyclohexenyl and cycloheptadienyl.

Unless otherwise indicated, the term “saturated or partially unsaturated C6 to 8 bicycloalkyl” referred to herein denotes a bicyclic alkyl group having from 6 to 8 carbon atoms and optionally incorporating one or more double bonds. Examples of such groups include bicyclo[2.2.1]heptenyl and bicyclo[2.2.2]octane.

Unless otherwise indicated, the term “benzo fused C4 to 8 cycloalkyl” referred to herein denotes a cyclic alkyl group having from 4 to 8 carbon atoms fused to a benzo ring. Examples of such groups include indanyl and 1,2,3,4-tetrahydronaphthalenyl.

Unless otherwise indicated, the term “C1 to 6 alkoxy” referred to herein denotes a straight or branched chain alkoxy group having from 1 to 6 carbon atoms. Examples of such groups include methoxy, ethoxy, 1-propoxy, 2-propoxy and tert-butoxy.

The term “C1 to 2 alkoxy” is to be interpreted analogously.

Unless otherwise indicated, the term “C1 to 6 alkylthio” referred to herein denotes a straight or branched chain alkyl group having from 1 to 6 carbon atoms that is bonded to the molecule via a sulphur atom. Examples of such groups include methylthio, ethylthio and propylthio.

Unless otherwise indicated, the term “C2 to 6 alkanoyl” referred to herein denotes a straight or branched chain alkyl group having from 1 to 5 carbon atoms bonded through a carbonyl group. Examples of such groups include acetyl, propionyl and pivaloyl.

Unless otherwise indicated, the term “halogen” referred to herein denotes fluoro, chloro, bromo and iodo.

Examples of an alkyl or alkoxy group optionally further substituted by one or more halogen atoms include CH₂Cl, CHCl₂, CCl₃, CH₂F, CHF₂, CF₃, CF₃CF₂, CF₃CH₂, CH₂FCH₂, CH₃CF₂, CF₃CH₂CH₂, OCF₃ and OCH₂CF₃.

Examples of a 5- or 6-membered heteroaromatic ring containing one or two heteroatoms independently selected from O, S and N include furan, thiophene, imidazole, thiazole, isoxazole, pyridine and pyrimidine.

Examples of a 5- or 6-membered saturated heterocyclic ring containing one or two heteroatoms independently selected from O, N and S include tetrahydrofuran, pyrrolidine, piperidine, morpholine, thiomorpholine and piperazine.

Examples of a 5 to 7-membered saturated or partially unsaturated heterocyclic ring containing one or two heteroatoms independently selected from O, N and S include tetrahydrofuran, pyrrolidine, pyrroline, imidazoline, tetrahydropyran, dehydropiperidine, piperidine, morpholine, thiomorpholine, perhydroazepine and piperazine.

Examples of a monocyclic or bicyclic heteroaromatic ring system containing one to three heteroatoms independently selected from O, N and S include furan, thiophene, imidazole, thiazole, isoxazole, pyridine, pyrimidine, indole, isoquinoline, benzofuran and benzothiadiazole.

Examples of a saturated 5- or 6-membered azacyclic ring optionally including one further heteroatom selected from O, S and N include pyrrolidine, morpholine, piperazine and piperidine.

Certain compounds of formula (I) are novel. A further aspect of the invention thus provides the following novel compounds of formula (I):
5-phenoxymethyl-4-(dihydrofuran-2-one-3-yl)-2,4-dihydro-[1,2,4]triazole-3-thione;
5-(2-chloro-phenoxymethyl)-4-(trans-2-hydroxycyclohexyl)-2,4-dihydro-[1,2,4]triazole-3-thione;
5-phenoxymethyl-4-(1,2,3,4-tetrahydro-naphthalen-1-yl)-2,4-dihydro-[1,2,4]triazole-3-thione;
4-(bicyclo[2.2.1]hept-5-en-2-yl)-5-pyridin-2-ylmethyl-2,4-dihydro-[1,2,4]triazole-3-thione;
4-(1-benzyl-pyrrolidin-3-yl)-5-pyridin-2-ylmethyl-2,4-dihydro-[1,2,4]triazole-3-thione;
4-((1R,2R)-2-benzyloxy-cyclopentyl)-5-pyridin-2-ylmethyl-2,4-dihydro-[1,2,4]triazole-3-thione;
   and pharmaceutically acceptable salts thereof.

A further aspect of the invention concerns the novel compounds of formula (I) for use as a medicament.

In a further aspect, the present invention provides novel compounds of formula (Ia) 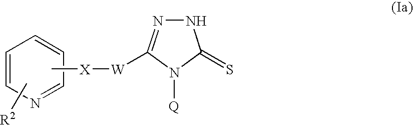
wherein:
Q represents a 5 to 7-membered saturated or partially unsaturated heterocyclic ring containing one or two heteroatoms independently selected from O, NR¹⁴ and S; said heterocyclic ring being optionally substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³ or NR⁴R⁵; said alkyl, cycloalkyl, alkoxy and alkylthio groups being optionally further substituted by phenyl or by one or more halogen atoms;
or Q represents C3 to 8 cycloalkyl substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³ or NR⁴R⁵; said alkyl, C3 to 6 cycloalkyl, alkoxy and alkylthio groups being optionally further substituted by phenyl or by one or more halogen atoms;
or Q represents partially unsaturated C5 to 8 cycloalkyl optionally substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³ or NR⁴R⁵; said alkyl, C3 to 6 cycloalkyl, alkoxy and alkylthio groups being optionally further substituted by phenyl or by one or more halogen atoms;
or Q represents saturated or partially unsaturated C6 to 8 bicycloalkyl optionally substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³ or NR⁴R⁵; said alkyl, C3 to 6 cycloalkyl, alkoxy and alkylthio groups being optionally further substituted by phenyl or by one or more halogen atoms;
and in each of the above definitions the ring Q is optionally benzo fused wherein the benzo ring is optionally substituted by one or more substituents independently selected from halogen, CHO, C2 to 6 alkanoyl, C1 to 6 alkyl, C1 to 6 alkylthio and C1 to 6 alkoxy;
or Q represents benzo fused C4 to 8 cycloalkyl wherein the benzo ring is optionally substituted by one or more substituents independently selected from halogen, CHO, C2 to 6 alkanoyl, C1 to 6 alkyl, C1 to 6 alkylthio and C1 to 6 alkoxy;
W represents CH₂;
X represents a bond;
R² represents H or one or more substituents independently selected from halogen, OH, C1 to 6 alkyl, C3 to 6 cycloalkyl, C1 to 6 alkoxy, C1 to 6 alkylthio, CO₂H, C2 to 6 alkanoyl, Ph, NO₂, C(O)NR¹²R¹³ or NR⁴R⁵; said alkyl, cycloalkyl, alkoxy and alkylthio groups being optionally further substituted by one or more fluoro atoms;
each R⁴, R⁵, R⁶, R¹² and R¹³ independently represents H or C1 to 6 alkyl;
R¹⁴ represents H, C1 to 6 alkyl, CHO or C2 to 6 alkanoyl; said alkyl being optionally further substituted by phenyl wherein the phenyl group may be optionally further substituted by halogen, C1 to 6 alkyl, C1 to 6 alkoxy or C2 to 6 alkanoyl;
and pharmaceutically acceptable salts thereof.

Particular compounds of formula (Ia) include:
4-(bicyclo[2.2.1]hept-5-en-2-yl)-5-pyridin-2-ylmethyl-2,4-dihydro-[1,2,4]triazole-3-thione;
4-(1-benzyl-pyrrolidin-3-yl)-5-pyridin-2-ylmethyl-2,4-dihydro-[1,2,4]triazole-3-thione;
4-((1R,2R)-2-benzyloxy-cyclopentyl)-5-pyridin-2-ylmethyl-2,4-dihydro-[1,2,4]triazole-3-thione;
   and pharmaceutically acceptable salts thereof.

A further aspect of the invention concerns the novel compounds of formula (Ia) for use as a medicament.

A further aspect of the invention concerns the novel compounds of formula (Ia) or a pharmaceutically acceptable salt thereof, in the manufacture of a medicament, for the treatment or prophylaxis of diseases or conditions in which inhibition of the enzyme MPO is beneficial.

According to the invention, we further provide a process for the preparation of the novel compounds of formula (I) or a pharmaceutically acceptable salt, enantiomer, diastereomer or racemate thereof which process [wherein variable groups are, unless otherwise specified, as defined in formula (I) above] comprises:
(a) reaction of a thiosemicarbazide derivative of formula (II) 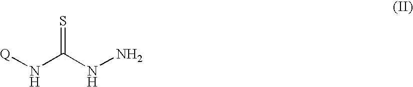
with an ester of formula (III) 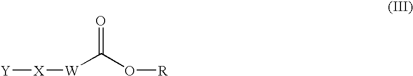
wherein R represents C1 to 6 alkyl; or
(b) reaction of a thiosemicarbazide derivative of formula (II),
with a carboxylic acid of formula (IV) 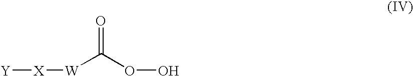
in the presence of a coupling agent; or
(c) reaction of a thiosemicarbazide derivative of formula (II),
with an acyl chloride of formula (V) 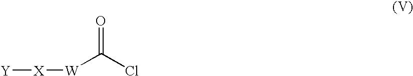
or
(d) reaction of an isothiocyanate derivative of formula (VI) 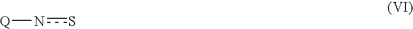
with an acid hydrazide of formula (VII) 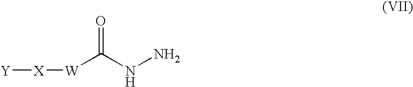
or
(e) reaction of an isocyanate derivative of formula (VIII) 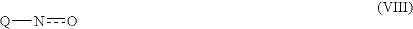
with an acid hydrazide of formula (VII) 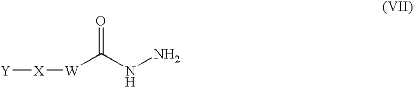
followed by treatment of the intermediate 2,4-dihydro-[1,2,4]triazol-3-one with Lawesson's reagent; or
(f) reaction of an dithioester derivative of formula (IX) 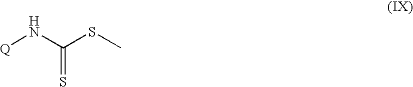
with an acid hydrazide of formula (VII) 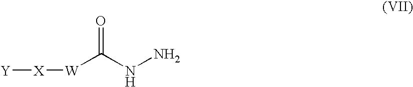
and where necessary converting the resultant compound of formula (I), or another salt thereof, into a pharmaceutically acceptable salt thereof; or converting the resultant compound of formula (I) into a further compound of formula (I); and where desired converting the resultant compound of formula (I) into an optical isomer thereof.

Compounds of formula (X) are key intermediates in the above processes

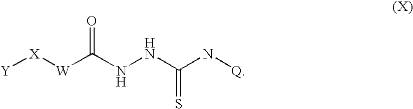

Depending on the conditions under which such intermediates are formed, and on the exact nature of the particular substituents Q, W, X and Y, such intermediates may be isolated or may undergo in situ cyclisation to give the compounds of formula (I). See, for example, Foks, H. et al. Phosphorus, Sulfur and Silicon, 2000, 164, 67-81; Udupi, R. H. et al. Indian Drugs, 2002, 39, 318-322; Pilgram, K. H. et al. J. Org. Chem., 1988, 53, 38-41; and Vidaluc, J-. L. et al. J. Med. Chem., 1994, 37, 689-695.

In process (a), the compounds of formulae (II) and (III) are reacted together in an organic solvent such as an alcohol, for example, methanol, in the presence of a base such as sodium methoxide, at a temperature between 25° C. and the reflux temperature of the reaction mixture until reaction is complete, typically for between 10 to 50 hours. See, for example, Pesson, M. et al. C.R. Hebd. Sceances Acad. Sci., 248; 1959; 1677-1679. The reaction mixture is then cooled and concentrated. The residue is dissolved in water and acidified with an acid such as acetic acid or hydrochloric acid, typically to pH about 3 to 6. The precipitate is collected and then purified by chromatography or recrystallization when necessary.

In process (b), the compounds of formulae (II) and (IV) are dissolved in an organic solvent such as dichloromethane, or DMF or mixtures thereof. A coupling reagent (for example, a peptide (amide) bond forming reagent) such as 1-(3-dimethylaminopropyl)-3-ethylcarbodiimide (EDC) is added at temperatures generally between 0 and 30° C. The reaction is stirred at temperatures between 10° C. and the reflux temperature of the solvent until the reaction is completed, typically for 1 to 15 h. The reaction mixture is concentrated and the residue is dissolved in a solvent, for example, a mixture of water and methanol with an added inorganic base such as sodium hydroxide or sodium hydrogen carbonate and heated to temperatures between 25° C. and the reflux temperature of the reaction mixture until the reaction is complete, typically for 30 minutes to 20 h. The reaction mixture is neutralized with an acid such as hydrochloric acid, and the precipitated product is collected by filtration. For reactions where the product does not precipitate, the reaction mixture is concentrated and the product is extracted with an organic solvent such as ethyl acetate or chloroform and the organic phase is dried and concentrated. The crude products are purified by chromatography or recrystallization when necessary.

In process (c), a compound of formula (V) in an organic solvent such as chloroform or dichloromethane containing a base such as pyridine or triethylamine is treated with a compound of formula (II). The reaction mixture is stirred at a temperature between 10° C. and the reflux temperature of the solvent until reaction is complete, typically for 1-16 h. The reaction mixture is concentrated and the residue is dissolved in a solvent such as water and methanol and the process is then continued as in process (b).

In process (d), the compounds of formulae (VI) and (VII) are dissolved in an organic solvent such as ethanol, isopropanol, DMF or dioxane or mixtures thereof, and then heated to between 25° C. and the reflux temperature of the solvent, preferably under an inert atmosphere until the reaction is completed, typically for 1 to 16 h. See, for example, Bamford, M. J. et al. J. Med. Chem. 1995, 38, 3502-3513; Abdelai, A. M. et al. Sci. Pharm. 1997, 65, 99-108; Petrovanu, M. Phosphorus, Sulphur and Silicon 1996, 108, 231-237. The reaction mixture is poured onto ice and the intermediate collected and, if necessary, purified by chromatography. If the intermediate does not precipitate, it is isolated by extraction with an organic solvent such as chloroform, ethyl acetate or diethyl ether. The intermediate is then dissolved in water or an alcohol or mixtures thereof, preferably with an added base such as, for example, sodium hydroxide or sodium hydrogen carbonate, and heated to between 25° C. and the reflux temperature of the solvent until the reaction is completed, typically for 1 to 16 h. The mixture is then neutralized by addition of an acid. Either the product precipitates upon neutralization, and it is then collected by filtration or the reaction mixture is extracted with an organic solvent. The crude product is then purified by chromatography or by recrystallization when necessary. In a particular embodiment, the compounds of formulae (VI) and (VII) are dissolved in an organic solvent such as ethanol, isopropanol, DMF or dioxane or mixtures thereof, and then heated in a microwave oven to a suitable temperature, generally between 120° C. and 150° C., for a suitable period of time, typically about 5 to 15 minutes. Under these conditions, the products of formula (I) may be formed directly without the need to isolate any intermediate.

In process (e), the compounds of formulae (VIII) and (VII) are reacted together using essentially the same conditions as for the reaction of compounds of formulae (VI) and (VII) in process (d), including in particular the use of microwave oven technology. The intermediate 2,4-dihydro-[1,2,4]triazol-3-one is then converted into the corresponding 2,4-dihydro-[1,2,4]triazole-3-thione of formula (I) by treatment with Lawesson's reagent. Suitable conditions for the use of Lawesson's reagent will be readily apparent to the man skilled in the art. See, for example, Cava, M. P. et al, Tetrahedron, 1985, 41, 5061-5087. Thus, for example, the intermediate 2,4-dihydro-[1,2,4]triazol-3-one and Lawesson's reagent are dissolved or suspended in a suitable dry organic solvent such as benzene, toluene, xylene, tetrahydrofuran, dichloromethane or dioxane and then heated to between 30° C. and the reflux temperature of the solvent until reaction is complete, typically for between one to 30 hours. If the sulphurisation reaction is conducted in a microwave oven, then suitable temperatures are generally between 120° C. and 150° C. and suitable reaction times are generally about 10 minutes to 1 hour.

In process (f), the dithioester of formula (IX) is dissolved in a suitable solvent such as absolute ethanol and the hydrazide (VII) is added. The reaction mixture is then heated at a suitable temperature, typically 80-90° C. for a suitable period of time, typically 3 to 16 h, prior to concentration and dissolution in methanol containing a base such as 2% aqueous NaOH. After heating at 70° C. for a suitable time, typically 2 to 21 h, the mixture is cooled, diluted with water and the pH adjusted to approx 7 with 1M HCl. The precipitate is collected and, if necessary, purified by crystallisation or by chromatography.

Compounds of formula (V) may be prepared by treatment of compounds of formula (IV) with thionyl chloride. See, for example, Encyclopaedia of Reagents for Organic Synthesis, Vol. 7, ed. Paquette, L. A., John Wiley & Sons, West Sussex, 1995.

The present invention includes compounds of formula (I) in the form of salts. Suitable salts include those formed with organic or inorganic acids or organic or inorganic bases. Such salts will normally be pharmaceutically acceptable although salts of non-pharmaceutically acceptable acids or bases may be of utility in the preparation and purification of the compound in question. Thus, preferred acid addition salts include those formed from hydrochloric, hydrobromic, sulphuric, phosphoric, citric, tartaric, lactic, pyruvic, acetic, succinic, fumaric, maleic, methanesulphonic and benzenesulphonic acids. Preferred base addition salts include those in which the cation is sodium, potassium, calcium, aluminium, lithium, magnesium, zinc, choline, ethanolamine or diethylamine.

Salts of compounds of formula (I) may be formed by reacting the free base, or a salt, enantiomer or racemate thereof, with one or more equivalents of the appropriate acid or base. The reaction may be carried out in a solvent or medium in which the salt is insoluble or in a solvent in which the salt is soluble, for example, water, dioxan, ethanol, tetrahydrofuran or diethyl ether, or a mixture of solvents, which may be removed in vacuo or by freeze drying. The reaction may also be a metathetical process or it may be carried out on an ion exchange resin.

Compounds of formulae (II), (III), (IV), (VI), (VII) and (VIII) are either known in the literature or may be prepared using known methods that will be readily apparent to the man skilled in the art.

The compounds of the invention and intermediates thereto may be isolated from their reaction mixtures and, if necessary further purified, by using standard techniques.

The compounds of formula (I) may exist in enantiomeric forms. Therefore, all enantiomers, diastereomers, racemates and mixtures thereof are included within the scope of the invention. The various optical isomers may be isolated by separation of a racemic mixture of the compounds using conventional techniques, for example, fractional crystallisation, or HPLC. Alternatively, the various optical isomers may be prepared directly using optically active starting materials.

Intermediate compounds may also exist in enantiomeric forms and may be used as purified enantiomers, diastereomers, racemates or mixtures.

The compounds of formula (I) and their pharmaceutically acceptable salts are useful because they possess pharmacological activity as inhibitors of the enzyme MPO.

The compounds of formulae (I) and their pharmaceutically acceptable salts are indicated for use in the treatment or prophylaxis of diseases or conditions in which modulation of the activity of the enzyme myeloperoxidase (MPO) is desirable. In particular, linkage of MPO activity to disease has been implicated in neuroinflammatory diseases. Therefore the compounds of the present invention are particularly indicated for use in the treatment of neuroinflammatory conditions or disorders in mammals including man. Such conditions or disorders will be readily apparent to the man skilled in the art.

Conditions or disorders that may be specifically mentioned include multiple sclerosis, Alzheimer's disease, Parkinson's disease, amyotrophic lateral sclerosis and stroke, as well as other inflammatory diseases or conditions such as asthma, chronic obstructive pulmonary disease, cystic fibrosis, idiopathic pulmonary fibrosis, acute respiratory distress syndrome, sinusitis, rhinitis, psoriasis, dermatitis, uveitis, gingivitis, atherosclerosis, inflammatory bowel disease, renal glomerular damage, liver fibrosis, sepsis, proctitis, rheumatoid arthritis, and inflammation associated with reperfusion injury, spinal cord injury and tissue damage/scarring/adhesion/rejection. Lung cancer has also been suggested to be associated with high MPO levels. The compounds are also expected to be useful in the treatment of pain.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

For the above mentioned therapeutic indications, the dosage administered will, of course, vary with the compound employed, the mode of administration and the treatment desired. However, in general, satisfactory results are obtained when the compounds are administered at a dosage of the solid form of between 1 mg and 2000 mg per day.

The compounds of formulae (I) and pharmaceutically acceptable derivatives thereof, may be used on their own, or in the form of appropriate pharmaceutical compositions in which the compound or derivative is in admixture with a pharmaceutically acceptable adjuvant, diluent or carrier. Administration may be by, but is not limited to, enteral (including oral, sublingual or rectal), intranasal, inhalation, intravenous, topical or other parenteral routes. Conventional procedures for the selection and preparation of suitable pharmaceutical formulations are described in, for example, “Pharmaceuticals—The Science of Dosage Form Designs”, M. E. Aulton, Churchill Livingstone, 1988. The pharmaceutical composition preferably comprises less than 80% and more preferably less than 50% of a compound of formulae (I) or a pharmaceutically acceptable salt thereof.

There is also provided a process for the preparation of such a pharmaceutical composition which comprises mixing the ingredients.

The invention is illustrated, but in no way limited, by the following examples:

General Methods

All solvents used were analytical grade and commercially available anhydrous solvents for reactions. Reactions were typically run under an inert atmosphere of nitrogen or argon.

¹H and ¹³C NMR spectra were recorded at 400 MHz for proton and 100 MHz for carbon-13 either on a Varian Unity+400 NMR Spectrometer equipped with a 5 mm BBO probe with Z-gradients, or a Bruker Avance 400 NMR spectrometer equipped with a 60 μl dual inverse flow probe with Z-gradients, or a Bruker DPX400 NMR spectrometer equipped with a 4-nucleus probe equipped with Z-gradients; or at 600 MHz for proton and 150 MHz for carbon-13, either on a Bruker DRX600 NMR Spectromete or a Bruker Avance 600 NMR spectrometer equipped with a 5 mm BBO probe with Z-gradients, a 5 mm TXI probe with Z-gradients or a 2.5 mm BBI probe with Z-gradients. Unless specifically noted in the examples, spectra were recorded at 400 MHz for proton and 100 MHz for carbon-13. The following reference signals were used: the middle line of DMSO-d₆ δ2.50 (¹H), δ 39.51 (¹³C); the middle line of CD₃OD δ 3.31 (¹H) or δ 49.15 (¹³C); acetone-d₆ 2.04 (¹H), 206.5 (¹³C); and CDCl₃ δ 7.26 (¹H), the middle line of CDCl₃ δ 77.16 (¹³C) (unless otherwise indicated).

Mass spectra were recorded on a Waters LCMS consisting of an Alliance 2795 (LC) and a ZQ single quadrupole mass spectrometer. The mass spectrometer was equipped with an electrospray ion source (ESI) operated in a positive or negative ion mode. The capillary voltage was 3 kV and the mass spectrometer was scanned from m/z 100-700 with a scan time of 0.3 or 0.8 s. Separations were performed on either Waters X-Terra MS, C8-columns, (3.5 μm, 50 or 100 mm×2.1 mm i.d.), or a ScantecLab's ACE 3 AQ column (100 mm×2.1 mm i.d.). The column temperature was set to 40° C. A linear gradient was applied using a neutral or acidic mobile phase system, running at 0% to 100% organic phase in 4-5 minutes, flow rate 0.3 ml/min. Neutral mobile phase system: acetonitrile/[10 mM NH₄OAc (aq)/MeCN (95:5)], or [10 mM NH₄OAc (aq)/MeCN (1/9)]/[10 mM NH₄OAc (aq)/MeCN (9/1)]. Acidic mobile phase system: [133 mM HCOOH (aq)/MeCN (5/95)]/[8 mM HCOOH (aq)/MeCN (98/2)]. Alternatively, mass spectra were recorded on a Finnigan MAT SSQ7000 equipped with a thermo spray ion source (TSP) operated in the positive mode and scanning from m/z 120-600 with a scan time of 1 s. Samples were introduced via an isocratic pump, Shimatzu LC-10AD. The mobile phase was 50 mM ammonium acetate in 40:60 acetonitrile/MilliQ Water and the flow rate 1 ml/min.

HPLC analyses were performed on an Agilent HP1000 system consisting of G1379A Micro Vacuum Degasser, G1312A Binary Pump, G1367A Wellplate auto-sampler, G1316A Thermostatted Column Compartment and G1315B Diode Array Detector. Column: X-Terra MS, Waters, 4.6×50 mm, 3.5 μm. The column temperature was set to 40° C. and the flow rate to 1.5 ml/min. The Diode Array Detector was scanned from 210-300 nm, step and peak width were set to 2 nm and 0.05 min, respectively. A linear gradient was applied, run from 0% to 100% acetonitrile, in 4 min. Mobile phase: acetonitrile/10 mM ammonium acetate in 5% acetonitrile in MilliQ Water.

A typical workup procedure after a reaction consisted in extraction of the product with a solvent such as ethyl acetate, washing with water followed by drying of the organic phase over MgSO₄ or Na₂SO₄ and concentration of the solution in vacuo.

Thin layer chromatography (TLC) was performed on Merck TLC-plates (Silica gel 60 F₂₅₄) and the spots were visualized by UV. Preparative layer chromatography was performed on Merck PLC-Plates (Silica gel 60 F₂₅₄, 2 mm). Merck Silica gel 60 (0.040-0.063 mm) was used for flash chromatography. Typical solvents used for flash chromatography were mixtures of chloroform/methanol, toluene/ethyl acetate and ethyl acetate/hexanes.

Preparative chromatography was run on a Gilson auto-preparative HPLC with a diode array detector. Column: XTerra MS C8, 19×300 mm, 7 μm. Gradient with acetonitrile/0.1M ammonium acetate in 5% acetonitrile in MilliQ Water, run from 20% to 60% acetonitrile, in 13 min. Flow rate: 20 ml/min. Alternatively, purification was achieved on a semi preparative Shimadzu LC-8A HPLC with a Shimadzu SPD-10A Uv-vis.-detector equipped with a Waters Symmetry® column (C18, 5 μm, 100 mm×19 mm). Gradient with acetonitrile/0.1% trifluoroacetic acid in MilliQ Water, run from 35% to 60% acetonitrile in 20 min. Flow rate: 10 ml/min.

Recrystallization was typically performed in solvents or solvent mixtures such as ether, ethyl acetate/heptanes and methanol/water.

The following abbreviations have been used: DMF=N,N-dimethylformamide; DMSO=dimethylsulfoxide; THF=tetrahydrofuran; EDC=1-(3-dimethylaminopropyl)-3-ethyl-carbodiimide hydrochloride; aq.=aqueous.

Starting materials used were either available from commercial sources or prepared according to literature procedures and had experimental data in accordance to those reported. The following are examples of starting materials that were prepared:
(2-Chlorophenyl)acetic acid hydrazide: Rosen, G. M. et al. J. Heterocycyl. Chem. 1971, 8, 659-662.
Phenoxyacetic acid hydrazide: Prata, J. V. et al. J. Chem. Soc., Perkin Trans 1, 2002, 4, 513-528.
2-Pyridylacetic acid hydrazide: Australian Journal of Chemistry, 1985, 38, 1491-1497.

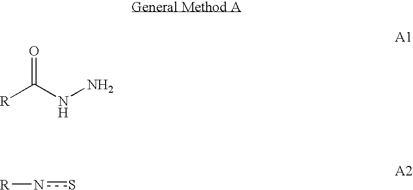

Compound A1 (1.0 equiv.) and compound A2 (1.5 to 2.5 equiv.) were dissolved in isopropanol and refluxed under an argon-atmosphere until the reaction was complete (monitored by LC-MS, HPLC or TLC; typical reaction times 1 to 21 h). The reaction mixture was cooled and poured onto ice and the precipitate was collected and washed with water. The precipitated intermediate was dissolved in 2% aqueous sodium hydroxide and refluxed for 2 to 12 h. The reaction mixture was cooled, neutralized with 1M hydrochloric acid and the precipitate was collected and purified if necessary by column chromatography or recrystallization.

Except where otherwise indicated, the compounds of Examples 1 to 5 were prepared using the procedure of General Method A.

### EXAMPLE 1

### 5-Phenoxymethyl-4-(dihydrofuran-2-one-3-yl)-2,4-dihydro-[1,2,4]triazole-3-thione

The title compound was obtained as a white foam in 22% yield starting from phenoxyacetic acid hydrazide (200 mg, 1.2 mmol) and 3-isothiocyanatodihydrofuran-2(3H)-one (258 mg, 1.8 mmol) using general procedure A with the following modifications. After treatment with 2% NaOH the mixture was acidified with HOAc, heated at 100° C. for three days, and then concentrated. The residue was dissolved in CH₂Cl₂ (10 mL) and DMF (2 mL), and EDC (230 mg, 1.2 mmol) was added. After 40 minutes, water was added and the mixture was extracted with CHCl₃. The organic phase was dried (Na₂SO₄), filtered, concentrated and purified by flash column chromatography (CHCl₃/MeOH, 50:1).

¹H NMR (DMSO-d₆, 340K) δ 13.88 (1H, s), 7.33 (2H, t, J=7.6 Hz), 7.03 (3H, m), 5.68 (1H, t, J=8.2 Hz), 5.20 (2H, m), 4.53 (1H, m), 4.42 (1H, m), 2.99 (1H, br s), 2.63 (1H, m). ¹³C NMR (DMSO-d₆, 340K) δ 170.6, 161.90, 156.7, 147.8, 129.2, 121.6, 114.8, 65.6, 60.0, 52.7, 24.9.

MS (ESI) m/z 292 (M+1).

### EXAMPLE 2

### 5-(2-Chloro-phenoxymethyl)-4-(trans-2-hydroxycyclohexyl)-2,4-dihydro-[1,2,4]triazole-3-thione

The title compound was obtained as a white solid in 18% yield starting from (2-chlorophenoxy)acetic acid hydrazide (179 mg, 1.08 mmol) and trans-cyclohexanol-2-isothiocyanate (170 mg, 1.08 mmol) using general procedure A with the following modifications. After the first step, the reaction mixture was concentrated in vacuo directly and not poured onto ice. The final product did not crystallize and was therefore extracted with EtOAc and purified by column chromatography (gradient elution 0-7% MeOH in CH₂Cl₂).

¹H NMR (CDCl₃) δ 7.37 (1H, dd, J=8 Hz, 1.2 Hz), 7.25 (1H, m), 7.12 (1H, m), 6.98 (1H, br t, J=7.4 Hz), 5.30 (2H, br s), 4.95 (1H, br s), 4.18 (1H, s), 2.81 (1H, br s), 2.22-2.12 (1H, m), 2.05-1.95 (1H, m), 1.89-1.73 (2H, m), 1.50-1.26 (4H, m).

MS (ESI) m/z 340 (M+1).

### EXAMPLE 3

### 5-Phenoxymethyl-4-(1,2,3,4-tetrahydro-naphthalen-1-yl)-2,4-dihydro-[1,2,4]triazole-3-thione

The title compound was obtained as white foam in 16% yield starting from phenoxyacetic acid hydrazide (669 mg, 2.77 mmol) and 1,2,3,4-tetrahydronaphthalene-1-isothiocyanate (655 mg, 3.47 mmol) using general procedure A with the following modifications. After the first step, the reaction mixture was concentrated directly and not poured onto ice. The residue was purified by flash chromatography (gradient elution 0-5% MeOH in CH₂Cl₂). The final product did not crystallize and was therefore extracted with EtOAc and purified by column chromatography (gradient elution 0-30% EtOAc in hexane).

¹H NMR (CDCl₃) δ 7.28-7.17 (4H, m), 7.13 (1H, t, J=7.2 Hz), 7.07 (1H, br d, J=7.2 Hz), 6.98 (1H, t, J=7.2 Hz), 6.86 (1H, br d, J=7.6 Hz), 6.72 (1H, m), 6.29 (1H, br s), 4.72 (1H, br s), 4.28 (1H, br s), 2.75 (2H, m), 2.42 (1H, m), 2.33-2.17 (1H, m), 2.12-2.02 (1H, m), 1.91-1.78 (1H, m).

¹³C NMR (CDCl₃) δ 156.9, 138.8, 132.4, 129.64, 129.58, 128.0, 127.0, 126.7, 122.0, 114.5, 60.3, 56.1, 29.1, 28.9, 21.9.

MS (ESI) m/z 338 (M+1).

### EXAMPLE 4

### 4-(Bicyclo[2.2.1]hept-5-en-2-yl)-5-pyridin-2-ylmethyl-2,4-dihydro-[1,2,4]triazole-3-thione

The title compound was obtained as a white solid in 36% yield starting from pyridine-2-yl-acetic acid hydrazide (0.151 g, 1.0 mmol) and 5-isothiocyanato-bicyclo[2.2.1]hept-2-ene (0.226 g, 1.5 mmol).

¹H NMR (DMSO-d6) δ 13.57 (1H, s), 8.56 (1H, d, J=4.4 Hz), 7.84 (1H, t, J=7.6 Hz), 7.41 (1H, d, J=7.6 Hz), 7.36 (1H, d, J=4.4 Hz), 6.22 (1H, s), 5.95 (1H, s), 4.38 (2H, s), 4.09 (1H, s), 3.29 (1H, m), 2.95 (1H, s), 2.70 (1H, d, J=8.4 Hz), 2.63 (1H, s), 1.42 (1H, t, J=10.0 Hz), 1.35 (1H, d, J=8.0 Hz).

¹³C NMR (DMSO-d6) δ 166.3, 155.6, 151.3, 149.3, 139.3, 137.1, 135.8, 123.4, 122.4, 58.9, 47.9, 46.9, 41.1, 34.6, 25.7.

MS (ESI) m/z 285 (M+1).

### EXAMPLE 5

### 4-(1-Benzyl-pyrrolidin-3-yl)-5-pyridin-2-ylmethyl-2,4-dihydro-[1,2,4]triazole-3-thione

Carbon disulfide (0.6 mL, 10 mmol) was added to a solution of 1-benzyl-pyrrolidin-3-ylamine (0.352 g, 2.0 mmol) and Et₃N (26 μL, 0.2 mmol) in THF (10 mL) and the mixture was stirred at 35° C. for 30 minutes. The mixture was cooled to 0° C. and 30% aqueous H₂O₂ (0.57 mL, 5.6 mmol) was added dropwise. The solution was acidified with 1M HCl and the mixture was diluted with water and extracted with EtOAc. The organic phase was dried (Na₂SO₄), filtered and concentrated in vacuo to give crude 1-benzyl-3-isothiocyanato-pyrrolidine.

The title compound was obtained as a white solid in 46% yield starting from pyridine-2-yl-acetic acid hydrazide (0.120 g, 0.79 mmol) and crude 1-benzyl-3-isothiocyanato-pyrrolidine using general method A.

¹H NMR (DMSO-d6) δ 8.43 (1H, d, J=4.55 Hz), 7.77 (1H, m), 7.38-7.18 (1H, m), 5.32-5.31 (1H, m), 4.69-4.53 (2H, m), 3.62-3.51 (2H, m), 3.07 (1H, dd, J=9.60 Hz, 5.31 Hz), 2.94 (1H, m), 2.60-2.42 (2H, m), 2.28-2.09 (2H, m).

¹³C NMR (DMSO-d6) δ 166.1, 156.2, 150.4, 149.1, 138.6, 136.9, 128.3, 128.1, 126.8, 123.2, 122.1, 59.0, 55.9, 53.6, 53.0, 33.6, 29.0.

MS (ESI) m/z 352 (+1).

### EXAMPLE 6

### 4-((1R,2R)-2-Benzyloxy-cyclopentyl)-5-pyridin-2-ylmethyl-2,4-dihydro-[1,2,4]triazole-3-thione

(1R,2R)-2-Benzyloxycyclopentylamine (0.38 g, 2.0 mmol) in EtOH (1.2 mL) was treated with triethylamine (280 μL, 2.0 mmol) followed by the dropwise addition of carbon disulfide (120 μL, 2.0 mmol). The suspension was stirred for 1 h, then methyl iodide (124 μL, 2.0 mmol) was added and the mixture was stirred at ambient temperature overnight. The mixture was concentrated, the oily residue was dissolved in EtOH (4 mL), and pyridin-2-yl-acetic acid hydrazide (0.51 g, 1.8 mmol) was added. The mixture was heated in a microwave reactor for 50 minutes at 150° C. The mixture was concentrated and the residue was dissolved in MeOH (8 mL) and 2% NaOH(aq) (4 mL) and heated to reflux for 4 h. The reaction was allowed to cool to ambient temperature and 2M HCl was added until pH 7. The MeOH was evaporated and the aqueous phase was extracted with CHCl₃. The organic phases were combined, dried (Na₂SO₄) and concentrated. The crude oil was purified by column chromatography (CHCl₃) to yield the title compound (35 mg, 6%). ¹H NMR (CDCl₃) δ ppm 11.92 (1H, br s), 8.46 (1H, m), 7.50 (1H, s), 7.25-7.15 (3H, m), 7.15-7.04 (4H, m), 5.05 (1H, dt, J=6.8 Hz, 14.0 Hz), 4.45 (1H, m), 4.39 (1H, d, J=12.4 Hz), 4.27 (1H, d, J=12.4 Hz), 4.22 (1H, d, J=17.2 Hz), 4.17 (1H, d, J=17.2 Hz), 2.65-2.52 (1H, m), 2.20-2.08 (1H, m) 1.93-1.80 (1H, m), 1.67-1.48 (3H, m).

MS (ESI) m/z 367 (M+1).

### Screens

Methods for the determination of MPO inhibitory activity are disclosed in patent application WO 02/090575. The pharmacological activity of compounds according to the invention was tested in the following screen in which the compounds were either tested alone or in the presence of added tyrosine:

Assay buffer: 20 mM sodium/potassium phosphate buffer pH 6.5 containing 10 mM taurine and 100 mM NaCl.

Developing reagent: 2 mM 3,3′,5,5′-tetramethylbenzidine (TMB), 200 μM KI, 200 mM acetate buffer pH 5.4 with 20% DMF.

To 10 μl of diluted compounds in assay buffer, 40 μl of human MPO (final concentration 2.5 nM), with or without 20 μM tyrosine (final concentration, if present, 8 μM), was added and the mixture was incubated for 10 minutes at ambient temperature. Then 50 μl of H₂O₂ (final concentration 100 μM), or assay buffer alone as a control, were added. After incubation for 10 minutes at ambient temperature, the reaction was stopped by adding 10 μl 0.2 mg/ml of catalase (final concentration 18 μg/ml). The reaction mixture was left for an additional 5 minutes before 100 μl of TMB developing reagent was added. The amount of oxidised 3,3′,5,5′-tetramethylbenzidine formed was then measured after about 5 minutes using absorbance spectroscopy at about 650 nM. IC₅₀ values were then determined using standard procedures.

When tested in at least one version of the above screen, the compounds of Examples 1 to 6 gave IC₅₀ values of less than 60 μM, indicating that they are expected to show useful therapeutic activity. Representative results are shown in the following Table.

| | Inhibition of MPO |
|---|---|
| Compound | (in the presence of tyrosine) IC₅₀ μM |
| Example 2 | 3.59 |
| Example 4 | 6.73 |
| Example 5 | 7.20 |

1. A method of treatment or prophylaxis of a disease or condition in which inhibition of the enzyme MPO is beneficial comprising administering to a patient in need thereof a compound of formula (I), or a pharmaceutically acceptable salt thereof, 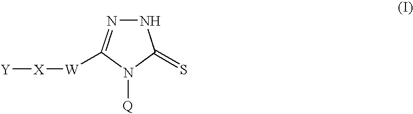
wherein:
Q is a 5 to 7-membered saturated or partially unsaturated heterocyclic ring containing one or two heteroatoms independently selected from O, NR¹⁴ and S; a C3 to 8 cycloalkyl; a partially unsaturated C5 to 8 cycloalkyl; a saturated or partially unsaturated C6 to 8 bicycloalkyl; or a benzo fused C4 to 8 cycloalkyl;
wherein said heterocyclic ring, C5 to 8 cycloalkyl; and C6 to 8 bicycloalkyl are optionally substituted by one to three substituents independently selected from halogen, OH, C1 to 6 alkyl C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³, and NR⁴R⁵; said alkyl C3 to 6 cycloalkyl, alkoxy and alkylthio being optionally substituted by phenyl or one or more halogen atoms;
wherein said C3 to 8 cycloalkyl is substituted by one to three substituents independently selected from halogen, OH, C1 to 6 alkyl C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³, and NR⁴R⁵; said alkyl, C3 to 6 cycloalkyl, alkoxy and alkylthio being optionally substituted by phenyl or one or more halogen atoms;
wherein the benzo ring of the benzo fused C4 to 8 cycloalkyl is optionally substituted by one or more substituents independently selected from halogen, CHO, C2 to 6 alkanoyl, C1 to 6 alkyl, C1 to 6 alkylthio, and C1 to 6 alkoxy;
further wherein said heterocyclic ring, said C3 to 8 cycloalkyl ring, said C5 to 8 cycloalkyl ring, and said C6 to 8 bicycloalkyl ring are each optionally fused to a benzo ring optionally substituted by one or more substituents independently selected from halogen, CHO, C2 to 6 alkanoyl, C1 to 6 alkyl, C1 to 6 alkylthio, and C1 to 6 alkoxy;
W is a bond or CHR₁, wherein R¹ is H, CH₃, F, OH, CH₂OH, or phenyl;
X is a bond, O, CH₂ or NR³, wherein R³ is H or C1 to 6 alkyl;
Y is phenyl, naphthyl, or a monocyclic or bicyclic heteroaromatic ring system containing one to three heteroatoms independently selected from O, N and S; wherein said phenyl, naphthyl or heteroaromatic ring system is optionally substituted by one to three substituents independently selected from halogen, OH, C1 to 6 alkyl C3 to 6 cycloalkyl, C1 to 6 alkoxy, C1 to 6 alkylthio, CO₂H, C2 to 6 alkanoyl, phenyl, NO₂, C(O)NR¹²R¹³, and NR⁴R⁵; said alkyl cycloalkyl, alkoxy and alkylthio being optionally substituted by one or more fluoro atoms; or
Y is C1 to 6 alkyl or C3 to 6 cycloalkyl; wherein said cycloalkyl optionally includes an O atom and is optionally benzo fused; and wherein said alkyl and cycloalkyl are optionally substituted by one or more substituents independently selected from halogen, oxo (═O), C1 to 6 alkyl, and C1 to 6 alkoxy;
R⁴, R⁵, R⁶, R¹² and R¹³ are each independently selected from H and or C1 to 6 alkyl;
R¹⁴ is H, C1 to 6 alkyl CHO or C2 to 6 alkanoyl; wherein said alkyl is optionally substituted by phenyl optionally substituted by halogen, C1 to 6 alkyl C1 to 6 alkoxy, or C2 to 6 alkanoyl.

2. The method according to claim 1 wherein the disease or condition is a neuroinflammatory disorder.

3. The method according to claim 1 wherein Y is an optionally substituted pyridyl.

4. The method according to claim 1, wherein Y is an optionally substituted phenyl.

5. The method according to claim 1, wherein W is a bond or CH₂.

6. The method according claim 1, wherein X is a bond or O.

7. A pharmaceutical formulation comprising a therapeutically effective amount of a compound, according to claim 1, or a pharmaceutically acceptable salt thereof, in admixture with a pharmaceutically acceptable adjuvant, diluent or carrier.

8. A compound selected from:
5-phenoxymethyl-4-(dihydrofuran-2-one-3-yl)-2,4-dihydro-[1,2,4]triazole-3-thione;
5-(2-chloro-phenoxymethyl)-4-(trans-2-hydroxycyclohexyl)-2,4-dihydro-[1,2,4]triazole-3-thione;
5-phenoxymethyl-4-(1,2,3,4-tetrahydro-naphthalen-1-yl)-2,4-dihydro-[1,2,4]triazole-3-thione;
4-(bicyclo[2.2.1]hept-5-en-2-yl)-5-pyridin-2-ylmethyl-2,4-dihydro-[1,2,4]triazole-3-thione;
4-(1-benzyl-pyrrolidin-3-yl)-5-pyridin-2-ylmethyl-2,4-dihydro-[1,2,4]triazole-3-thione; and
4-((1R,2R)-2-benzyloxy-cyclopentyl)-5-pyridin-2-ylmethyl-2,4-dihydro-[1,2,4]triazole-3-thione;
or a pharmaceutically acceptable salt thereof.

9. A pharmaceutical formulation comprising a therapeutically effective amount of a compound according to claim 8, or a pharmaceutically acceptable salt thereof, in admixture with a pharmaceutically acceptable adjuvant, diluent or carrier.

10. A compound of formula (Ia)), or a pharmaceutically acceptable salt thereof, 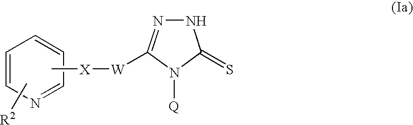
wherein:
Q is a 5 to 7-membered saturated or partially unsaturated heterocyclic ring containing one or two heteroatoms independently selected from O, NR¹⁴ and S; a C3 to 8 cycloalkyl; a partially unsaturated C5 to 8 cycloalkyl; a saturated or partially unsaturated C6 to 8 bicycloalkyl; or a benzo fused C4 to 8 cycloalkyl;
wherein said heterocyclic ring, C5 to 8 cycloalkyl; and C6 to 8 bicycloalkyl are optionally substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³, and NR⁴R⁵; said alkyl, C3 to 6 cycloalkyl, alkoxy and alkylthio being optionally substituted by phenyl or none or more halogen atoms;
wherein said C3 to 8 cycloalkyl is substituted by one to three substituents independently selected from halogen, OH, C1 to 6 alkyl, C3 to 6 cycloalkyl, C1 to 6 alkoxy, C1 to 6 alkylthio, oxo (═O), CO₂R⁶, CHO, C2 to 6 alkanoyl, phenyl, NO₂, C(O)NR¹²R¹³, and NR⁴R⁵; said alkyl, C3 to 6 cycloalkyl, alkoxy and alkylthio being optionally substituted by phenyl or one or more halogen atoms;
wherein the benzo ring of the benzo fused C4 to 8 cycloalkyl is optionally substituted by one or more substituents independently selected from halogen, CHO, C2 to 6 alkanoyl, C1 to 6 alkyl, C1 to 6 alkylthio, and C1 to 6 alkoxy;
further wherein said heterocyclic ring, said C3 to 8 cycloalkyl ring, said C5 to 8 cycloalkyl ring, and said C6 to 8 bicycloalkyl ring are each optionally fused to a benzo ring optionally substituted by one or more substituents independently selected from halogen, CHO, C2 to 6 alkanoyl, C1 to 6 alkyl, C1 to 6 alkylthio, and C1 to 6 alkoxy;
W is CH₂;
X is a bond;
R² is H, halogen, OH, C1 to 6 alkyl, C3 to 6 cycloalkyl, C1 to 6 alkoxy, C1 to 6 alkylthio, CO₂H, C2 to 6 alkanoyl, Ph, NO₂, C(O)NR¹²R¹³ or NR⁴R⁵; wherein said alkyl, cycloalkyl, alkoxy and alkylthio are optionally substituted by one or more fluoro atoms;
R⁴, R⁵, R⁶, R¹² and R¹³ are each independently selected from H and C1 to 6 alkyl;
R¹⁴ is H, C1 to 6 alkyl, CHO or C2 to 6 alkanoyl; wherein said alkyl is optionally substituted by a phenyl optionally substituted by halogen, C1 to 6 alkyl, C1 to 6 alkoxy or C2 to 6 alkanoyl.

11. (canceled)

12. A pharmaceutical composition comprising a compound according to claim 8, or a pharmaceutically acceptable salt thereof, in admixture with a pharmaceutically acceptable adjuvant, diluent or carrier.

13. (canceled)

14. A process for preparing a compound of formula (Ia), or a pharmaceutically acceptable salt, enantiomer, diastereomer or racemate thereof, 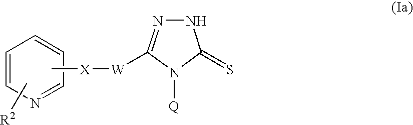
comprising:
I. (a) reacting a thiosemicarbazide derivative of formula (II) 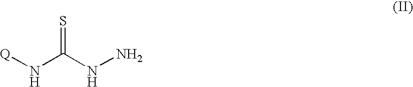
with
(i) an ester of formula (III) 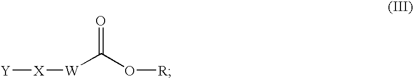
(ii) a carboxylic acid of formula (IV) 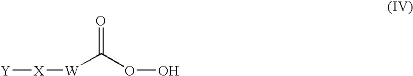
in the presence of a coupling agent; or
(iii) an acyl chloride of formula (V) 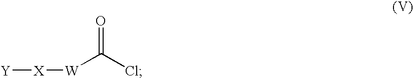
wherein R represents C1 to 6 alkyl and Q, Y, X, and W are as defined in claim 10 hereinabove; or
(b) reacting an isothiocyanate derivative of formula (VI) 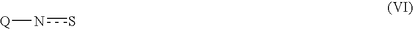
with an acid hydrazide of formula (VII) 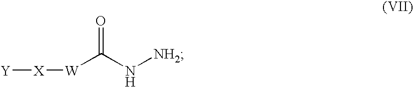
wherein Q, Y, X, and W are as defined in claim 10 hereinabove; or
(c) reacting an isocyanate derivative of formula (VIII)
Q-N [symbol] O (VIII)
with an acid hydrazide of formula (VII) 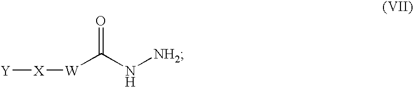
wherein Q, Y, X, and W are as defined in claim 10 hereinabove, followed by treating the intermediate 2,4-dihydro-[1,2,4]triazol-3-one with Lawesson's reagent; or
(d) reacting a dithioester derivative of formula (IX) 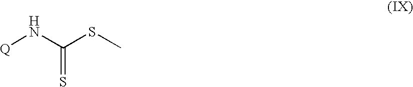
with an acid hydrazide of formula (VII) 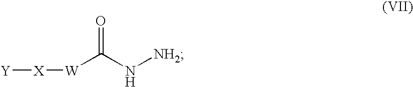
wherein Q, Y, X, and W are as defined in claim 10 hereinabove;
II. (a) optionally converting the resulting compound of formula (Ia), or a salt thereof, into a pharmaceutically acceptable salt thereof; or
(b) converting the resulting compound of formula (Ia) into a further compound of formula (Ia); and
III. optionally converting the resulting compound of formula (Ia) into an optical isomer thereof.
